(19) United States
(12) Patent Application Publication
(10) Pub. No.: US 2009/0000616 A9
(43) Pub. Date: Jan. 1, 2009

(54) Nasal devices including dilation and user communication and methods of using same

(76) Inventor: Gustav R. Fenton, Minneapolis, MN (US)

Correspondence Address: John F. Klos, Esq.;Fulbright & Jaworski LLIP, 2100 IDS Center, 80 South 8th Street, Minneapolis, MN, 55402-2112, US

(21) Appl. No.: 10/883,064

(22) Filed: Jun. 30, 2004

Related U.S. Application Data
(63) Continuation of application No. PCT/US02/41505, filed on Dec. 27, 2002.
(60) Provisional application No. 60/346,071, filed on Dec. 31, 2001.

(51) Int. Cl. A62B 7/00; A61M 15/00; A61M 16/00

(52) U.S. Cl. 128/200.24; 128/207.18

(57) ABSTRACT

An apparatus and methods of providing dilation to external tissue and user communication via a nasal device are provided. One embodiment comprises a flexible strip of material adapted to be adhesively secured to nasal surfaces of a user, and a signal unit which receives physiological signals of the user while disposed upon the nasal surfaces and which communicates information to the user via one or more of: acoustic vibrations, tactile contact and light emission. One or more signal units may be provided. The signal units may detect physiological parameters of the user. The signal unit may wirelessly communicate information relating to use to an external receiver. The signal unit may receive commands via wireless communication to control a subsequent communication to the user via vibrations, contact, and/or light emission. Additional embodiments comprise a fluid-filled elongated vessel adapted to conform to nasal surfaces of the user in order to dilate nasal regions. The fluid-filled vessel may include pressurized air. Another embodiment comprises a reusable resilient member engaging an elongated member having a pair of pockets disposed at each end of the elongated

### RELATED APPLICATIONS

This application claims the benefit of priority of PCT/US02/41505, filed Dec. 27, 2002, and U.S. Provisional Application Ser. No. 60/346,071, filed Dec. 31, 2001.

### FIELD OF THE INVENTION

The present invention relates to apparatus for and methods of influence surface tissue for therapeutic and/or aesthetic reasons. In particular, the present invention is directed to discrete embodiments and techniques for sensing, signaling, and/or dilating tissue proximate a nasal passage using an external device.

### BACKGROUND OF THE INVENTION

The field of endeavor related to dilation of nasal passages and adjacent tissue using over the nose-type dilator devices has a short and active history. One active participant and innovator in this field is the present assignee, CNS, Inc. (CNS) of Eden Prairie, Minn.

The disposable over the nose dilator devices of the prior art provide sufficient albeit rudimentary dilation of tissue adjacent nasal passageways and thus provide a modicum of increased respiration and relief from snoring in the vast majority of users. However, these prior art designs are generally not adjustable by a user, and do not incorporate additional functionality for, or generate additional benefits to, the user.

Thus, a need in the art exists for continued innovation and greater functionality for nasal devices. For example, a need exists for dilator devices that are simple to fabricate, that effectively dilate tissue, that may be adjusted in length and magnitude of lifting force imparted to the tissue, that may be accurately aligned relative to the tissue, that are more easily removed from the tissue, that are reusable and which are, in general, more comfortable to the user than prior art dilator devices. Additionally, a need exists for a nasal-mounted device having a signal unit for detecting nasal vibrations or other signals indicative of physiological functions of the user. The signal unit may provide biofeedback to a user to assist in the control of breathing to, for example, assist in control or prevention of panic attacks, facilitate meditations, etc.

### SUMMARY OF THE INVENTION

The present invention teaches, enables, illustrates, describes and claims new, useful and non-obvious apparatus and methods of providing dilation and/or signaling to external tissue. The present invention builds upon prior commercial embodiments of tissue dilator devices and addresses several still unmet needs in the art of fabricating, aligning, adjusting, applying, using, re-using and/or removing nasal tissue devices.

On particular invention disclosed herein provides a nasal device having a flexible strip of material adapted to be adhesively secured on nasal surfaces of a user and to provide tactile communication to the user at the nasal surfaces. One embodiment of the invention may include a sensor for detecting one or more physiological parameters of the user. In an embodiment of the present invention the tactile communication may be controlled in response to the sensed physiological information. In an embodiment of the present invention, an acoustic sensor is used to sense vibrations of the nasal surfaces related to the user's breathing pattern. Other embodiments of the present may include multiple sensors and/or multiple means to communicate to the user via the nasal surface.

In particular, without limitation, certain other inventions herein relate to a family of nasal dilator devices that: may be fabricated with tissue-protective qualities; may be applied (i.e., fabricated in situ) by a user; have a user-selectable magnitude of adhesion; a user-adjustable length and magnitude of desirable lifting force imparted by the dilator device; may be used to delivery a wide variety of scents and/or medications to the user; provide biofeedback to a user; have parts that may be re-used and parts that are used only once; and/or are readily manually removed from tissue without needlessly stressing such tissue.

The several embodiments of the present invention are described with reference to examples of forms of the invention comprehended by the devices taught, enabled, described, illustrated and claimed herein but all structures and methods which embody similar functionality are intended to be covered hereby. These embodiments include without limitation the following numbered, discrete forms of the invention, as more fully developed in the detailed description appearing hereinbelow.

### BRIEF DESCRIPTION OF THE DRAWINGS

In the drawings which accompany this disclosure, like elements are referred to with common reference numerals. The drawings are not rendered to scale and illustrate only a few of the many, many embodiments of tissue dilators which may be created according to the teaching of the present invention.

FIG. 1 is perspective view of the embodiment of the present invention shown as placed upon nasal surfaces of a user.

FIG. 2 is a perspective view of an embodiment of a nasal dilator according to the present invention wherein at least one signal unit is integrated into said nasal dilator either at an adhesive pad location or in the base member of said nasal dilator.

FIG. 3 is a simplified depiction of one type of signal unit which may be incorporated into a nasal surface contacting device.

FIG. 4 is a plan view of an embodiment of the present invention having two opposing pocket features formed in an upper surface of a nasal dilator device.

FIG. 5 is an elevational side view in cross section taken along the lines 5-5 of FIG. 4 of the embodiment of the nasal dilator depicted in FIG. 4.

FIG. 6 depicts an elevational side view in cross section of another embodiment of the present invention depicted in FIG. 4 and FIG. 5 wherein two opposing pocket features are each separate pieces spaced apart and adapted to receive at least one resilient member (shown in FIG. 7).

FIG. 7 is a perspective view of several resilient members usable with the embodiments of FIGS. 4-6.

FIG. 8 is a plan view of an embodiment of the present invention which is designed and configured with an intermediate portion which provides an increased tissue dilation lifting force at each end when one end is rotated relative to the other end and both ends are subsequently attached to tissue to be dilated.

FIG. 9 is a plan view of an embodiment of the present invention wherein the base portion of the nasal dilator is formed from a resilient scrim, or mesh, material and wherein one side of said nasal dilator has adhesive disposed thereon.

FIG. 10 is an elevational side view of a nasal dilator wherein the nasal dilator further comprises a pair of adhesive pads which are adhered to user-adjustable, spaced apart locations on one side of the nasal dilator and further depicting an optional spacer member adhered on one side to the middle region of the nasal dilator to thereby provide an increased dilating lifting force and to provide added comfort to the user.

FIG. 11 is an illustration of a nasal dilator which is fabricated in situ on tissue of a user and wherein the nasal dilator comprises a scrim, or mesh, material which is coated with an adhesive material.

FIG. 12 is a plan view of an elongated nasal dilator displaying several release mechanisms used to promote removal of the nasal dilator from tissue of a user.

FIG. 13 is an elevational side view of an embodiment of a nasal dilator according to the present invention wherein a layer of adhesive material is disposed on a side of the nasal dilator and said layer of adhesive material is selectively activated by mechanical action to effectively increase the amount of adhesive available to adhere the nasal dilator to tissue of a user.

FIG. 14a to FIG. 14d depict a family of nasal dilators comprised of an elongated unit sealed to ambient conditions and inflated, or filled, with a fluid material—this type of dilator device optionally has a resilient member coupled to an inner and/or an outer surface of the dilator device, a valve for increasing or decreasing the internal fluid pressure and a pair of pads adapted to adhere to a user's nose.

### DETAILED DESCRIPTION OF THE PREFERRED EMBODIMENTS

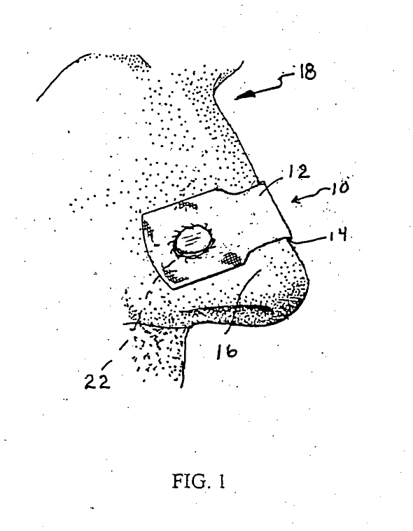

FIG. 1 illustrates in perspective view one embodiment of a nasal device 10 according to the present invention. Nasal device 10 includes a strip of flexible material 12 and an adhesive 14 for securing nasal device 10 to nasal surfaces 16 of a user 18. A resilient member 20 may be provided to create lift to the nasal surfaces 16. Other embodiments of the present invention may not require resilient means 20 to provide lift to the nasal surfaces 16. Nasal device 10 further includes a signal unit 22 for providing communication to user 18 during usage of device 10.

Particular selection of a flexible material 12, adhesive 14, and resilient member 20 would be appreciated by those skilled in the art, particularly with reference to U.S. Pat. Nos. 5,476,091; 5,533,499; 5,494,103; 5,653,224; 6,318,362; 6,196,228; 6,354,436; 5,546,929; 5,553,605; 5,718,224; and 5,479,944, each patent being incorporated by reference herein for all purposes.

Now with reference to FIG. 2 which is a perspective view of an embodiment of a nasal dilator 10 according to the present invention wherein at least one signal unit 22 is integrated with a nasal dilator 10. Signal unit 22 may be disposed at other locations, and multiple signal units 22 may be utilized. One or more of the signal units 22 may be affixed to the dilator 10. For example, a single signal unit 22 may be disposed at or near a midpoint of the dilator 10 so that firm contact is established with the bridge of the nose of the user or may be disposed in a location surrounded by, or nearly surrounded by, adhesive material. Signal unit 22 may be incorporated with dilator 10 at the time of manufacture or may be a removable device which is reusable by the user.

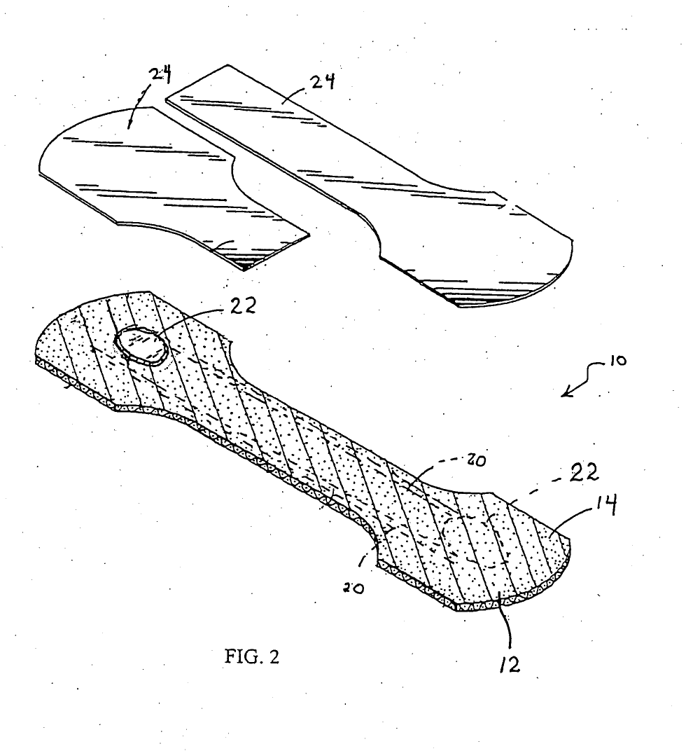

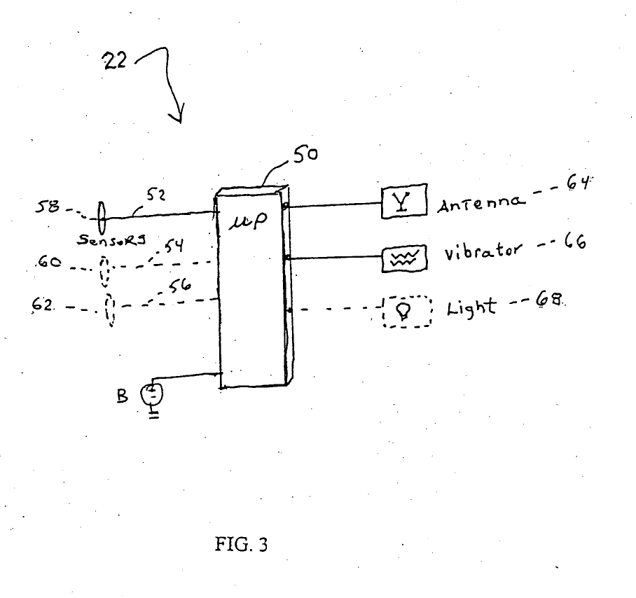

Release liners 24 may be removed to expose adhesive 14. Referring now to FIG. 3, signal unit 22 may include a microprocessor 50 which receives signals 52, 54, 56 from one or more sensors 58, 60, 62 and controls operation of functions such as communication, mechanical vibration, light emission, etc. Sensor 58 may be an acoustic sensor for sensing vibrations of nasal tissue indicative of a user's breathing patterns. A variety of known acoustic sensor may be utilized, for example, acoustic sensor may be piezoelectric device, etc. One or more sensor may be provided. Other sensor technologies may also be applicable. Microprocessor 50 may collect signal data from sensors 58, 60, 62 and perform data computations to control one or more outputs 64, 66, 68. Microprocessor 50 may include a memory means for storing collected data from nasal sensors 58, 60, 62. Microprocessor 50 may control a wireless communication device 64 including a transceiver and an antenna for communicating information to a user or another. Communication device 64 may receive or transmit via radio-frequency, infrared, or light-based communication. Communication device 64 may transmit a signal which is received by another antenna and subsequently processed by an external data processor. Communication unit 64 may also provide for signal reception to control a function of microprocessor 50. Communication device 64 may be integrated with microprocessor 50. Microprocessor 50 may also control operation of a mechanical vibrator 66 to signal a user. Mechanical vibrator 66 may be a piezoelectric device, for example. The function of vibrator 66 is to produce tactile and/or acoustic communication to a user of the nasal device. Vibrator 66 may apply a periodic pulse signal which is detectable by the user and which may be inaudible. For example, such an inaudible tactile pulse may be detected simply due to the contact between nasal tissue 46 and signal unit 22. In another embodiment, vibrator 66 may also produce an audible signal. Vibrator 66 may simply transmit a small impulse or series of impulses to a user, and not otherwise vibrate for a period of time. In this sense, vibrator 66 may or may not transmit a cyclic response to user.

Microprocessor 50 may control vibrator 66 to provide a “pacing” function which may help a user relax or compare their own present rate of respiration to a referenced respiration rate; for example during a panic attack or hyperventilation. Microprocessor 50 may function to receive an input corresponding to the current respiration rate and output a signal to the user via vibrator 66 indicative of a desired respiration rate (which may be higher or lower than the current rate) by reference to a look-up table or by other calculations, etc. The signal unit 22 may also help a user meditate or increase a level of concentration due to application of rhythmic pulses. In a related embodiment, the rate of the pulses produced by the vibrator 66 may be adjusted by the user to an increased tempo to promote a greater rate of respiration or movement of the user. Such an adjustable timing function may also be used to simply allow the user to set a comfortable pace, so that, for example, when the user is engaged is competition or sports events the user can sense when the difference between a quiescent state and an excited state and may more rapidly transition from one state to the other state. In another embodiment, signal unit 22 may detect voice commands of the user to control operation of vibrator 66 or other user signaling means. Signal unit 22 may receive additional signals from sensors 60, 62. For example, other physiological conditions such as a heart rate parameter, a blood pressure parameter, or a temperature parameter may be detected and utilized alone or in conjunction with breathing information as inputs to microprocessor 50. Signal unit 22 may also control light device 68 for communicating information related the input signals 52, 54, 56 to the user.

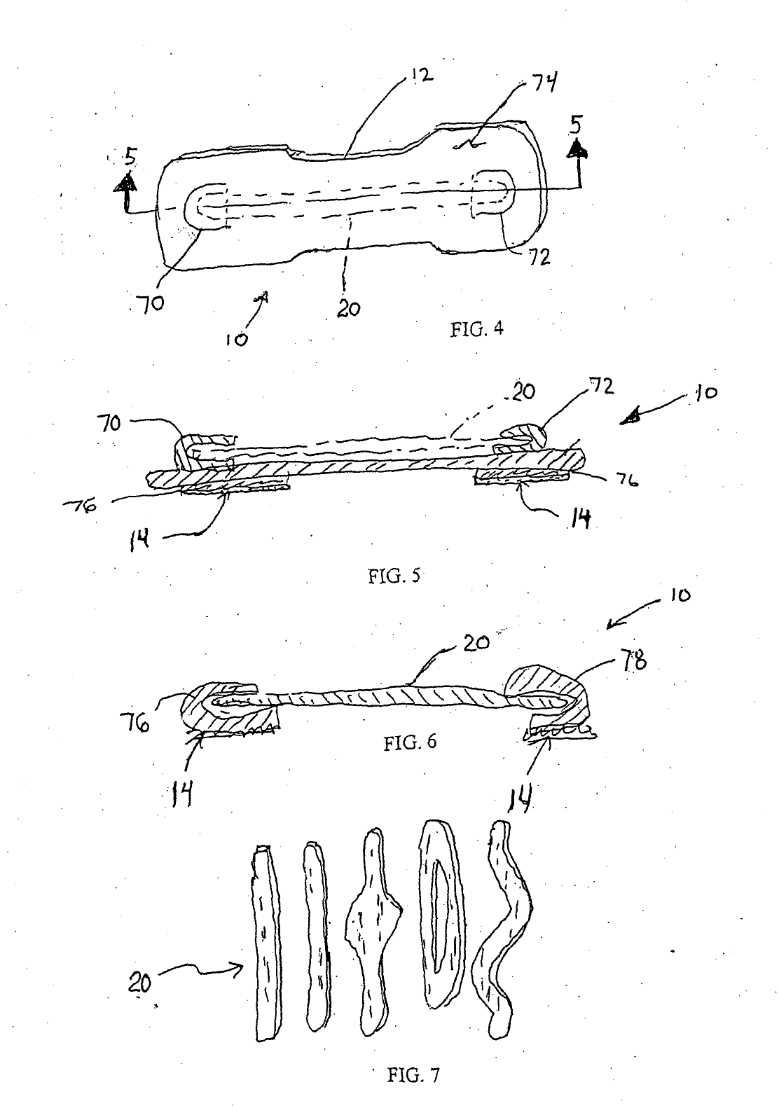

Referring now to FIG. 4, which is a plan view of an embodiment of a nasal dilator 10 of the present invention which comprises an elongated base member of flexible material 12 having two opposing pocket features 70, 72 formed in an upper major surface 74 of the nasal dilator 10. A pair of patches, or a layer, of adhesive 14 (shown in FIG. 5 and FIG. 6) is disposed on a lower surface to adhesively couple the dilator 10 to tissue of a user (not shown). Pockets 70, 72 are configured to receive at least one resilient member 20 (shown in ghost in FIG. 4) which essentially comprise an elongated member composed of a material which generates a restoring force. The restoring force tends to resist such bending so that when the ends of the resilient member 20 are constrained in the pockets 70, 72 tissue underlying the adhesive 14 receives said restoring force as a lifting force more or less directed orthogonal to the surface of the tissue to promote expansion of underlying nasal passages.

As shown in FIG. 5, which is an elevational side view in cross section taken along the lines 5-5 of FIG. 4, the pockets 70, 72 may have a pair of openings facing each other or may comprise an open-ended strap feature which captures the lifting force provided by the resilient member 20 so that the pads 76 which are adhered to the tissue imparts substantially all said lifting force orthongonally to said tissue. In the embodiment depicted in FIG. 4 and FIG. 5 the pockets 70, 72 appear as individual parts vis-a-vis the base 12 and the pads 76 but these parts may be fabricated as a single integral unit of construction. This form of the invention is inherently at least partially reusable in that the resilient member 20 may be retrieved from the assembly and used with another structure having the combination of a base 12, pockets 70, 72 and pads 76.

FIG. 6 depicts an elevational side view in cross section of form of the present invention depicted in FIG. 4 and FIG. 5 except that the two opposing pocket features are each separate parts spaced apart, supported on the tissue and adapted to receive at least one resilient member 20. In fact, this special form of the present invention is an embodiment having two discrete structures which each corresponding only to a single pocket and pad pair and wherein a resilient member 20 bridges between them. One advantage to forms of the present invention such as these is not only the ability to reuse the resilient member 20, but also that the resilient member 20 may be selected from a plurality of resilient members 20 each providing a slightly different magnitude of lifting force or configuration.

As illustrated in FIG. 7 is a plan view of several different resilient members 20 usable according to the present invention. The resilient members 20 may be used individually or combined with other of said resilient members 20 as desired. In addition to the shapes depicted the resilient members may have an undulating or serpentine cross sectional shape (e.g., analogous to the letters “W” or “U” and the like). In addition, a resilient member 20 usable with most forms of the present invention forms a geometric shape in lateral cross section and includes internally hollow and perforated forms of such resilient members 20, so long as the resilient member 20 so constructed provides the required lifting force to the tissue. Furthermore, in several adjustable-length embodiments of the present invention, resilient members 20 of all shapes may advantageously be partially perforated and manually shortened to suit a particular desired length or cut with an implement such as a scissor, knife or other sharpened blade and the like.

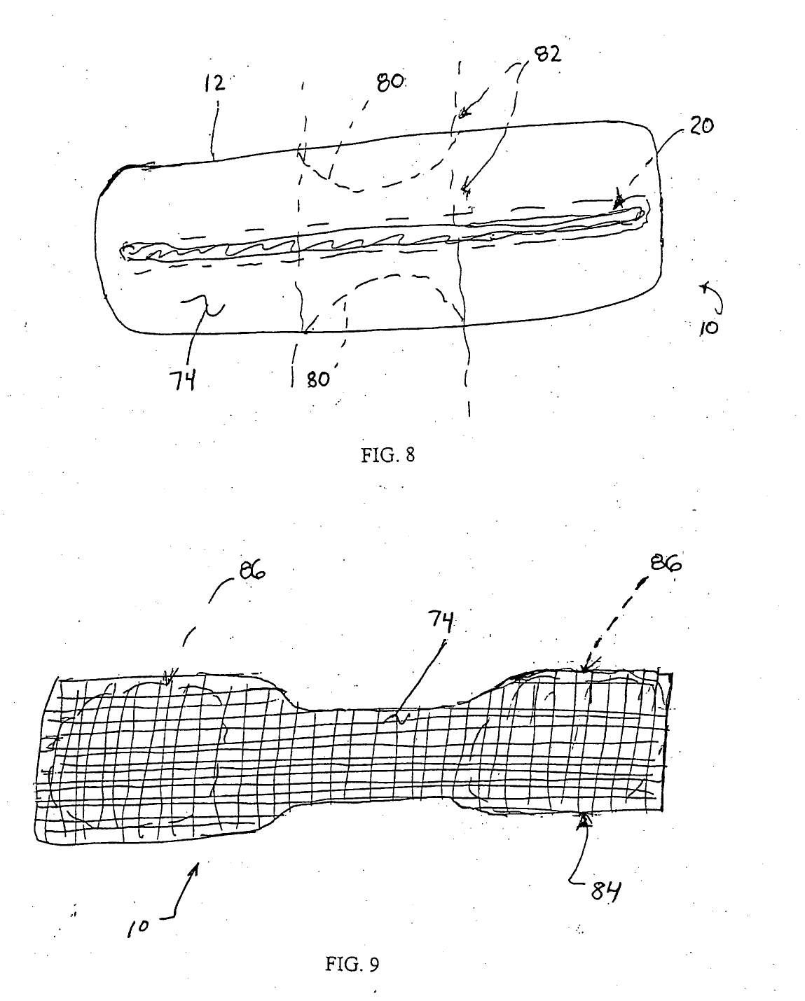

FIG. 8 is a plan view of an embodiment of a nasal dilator 10 according to the present invention which is designed and configured with an intermediate portion 22 in the base 12 (shown in ghost in FIG. 8). When the base 12 is twisted about the intermediate portion 22 the base 12 effectively shortens in length for every turn of the base 12 of the dilator 10. Also, the twisting action thereby incidentally increases the magnitude of the tissue dilation lifting force at each end of the dilator 10 when both ends are subsequently attached to tissue to be dilated. In this form of the invention, a resilient member 20 is preferably disposed axially through the intermediate portion 22 without binding to the base 12. In this regard, an elongated pocket feature 16 may be formed in the base 12 to loosely accommodate the resilient member 20 therein. Thus, when the ends of the base 12 are rotated, the resilient member 20 is not twisted. In this form of the invention either or both major surfaces may have a layer of adhesive disposed thereon. Of course, if only major surface has such a layer of adhesive, then only integer turns of the ends of the dilator 10 may be utilized such that the end of each adhesive-bearing major surface can adhere to the tissue. As shown also in ghost in FIG. 8 (as numeral 80), peripheral or lateral cutouts 80 may be removed from the base 12 to promote a tighter, more uniform cross sectional surface when the base 12 is twisted about the intermediate portion 82. While not depicted in FIG. 8, added material in the form of additional layers of material, or a section of mesh or scrim material, may be added to the intermediate portion 82 to at the same time strengthen the intermediate portion 82 and to provide a greater binding force (and thereby a greater lifting force) when the intermediate portion 82 is twisted and applied to tissue. Another form of this embodiment comprises a base 12 having a release liner (not shown in FIG. 8) covering adhesive portions disposed on each major surface and therefore may be reused at least once simply by removing one set of release liners and applying as indicated above. Thereafter, a user simply removes the second set of release liners and applies the dilator 10 so that the adhesive underlying the second set of release liners is adhered to the tissue.

Referring now to FIG. 9 which is a plan view of an embodiment of the present invention wherein the base portion of the nasal dilator 10 is formed from a resilient scrim, or mesh, material 28 and wherein one major surface of said nasal dilator 10 has adhesive disposed on a portion 86 thereof. The resilient scrim or mesh material 84 may be a woven or blown fiber material or otherwise form a perforated base. Preferably the resilient scrim or mesh material 84 possesses a restoring force when deflected so that when adhered to tissue, the material 84 provides a lifting force to the tissue. Regardless, the material 84 may be augmented with at least one elongated resilient member 20 anchored to its base as described elsewhere in this disclosure. On the major surface which is disposed adjacent the tissue, portions 86 are preferably provided with an adhesive applied thereto either a pad member (not shown) or directly to the material 84. This embodiment has several advantages, including lighter weight relative to known tissue dilators and the fact that increased air flow under, around and through the dilator 10 readily occurs thereby cooling the tissue and generally rendering the dilator 10 more comfortable to the user.

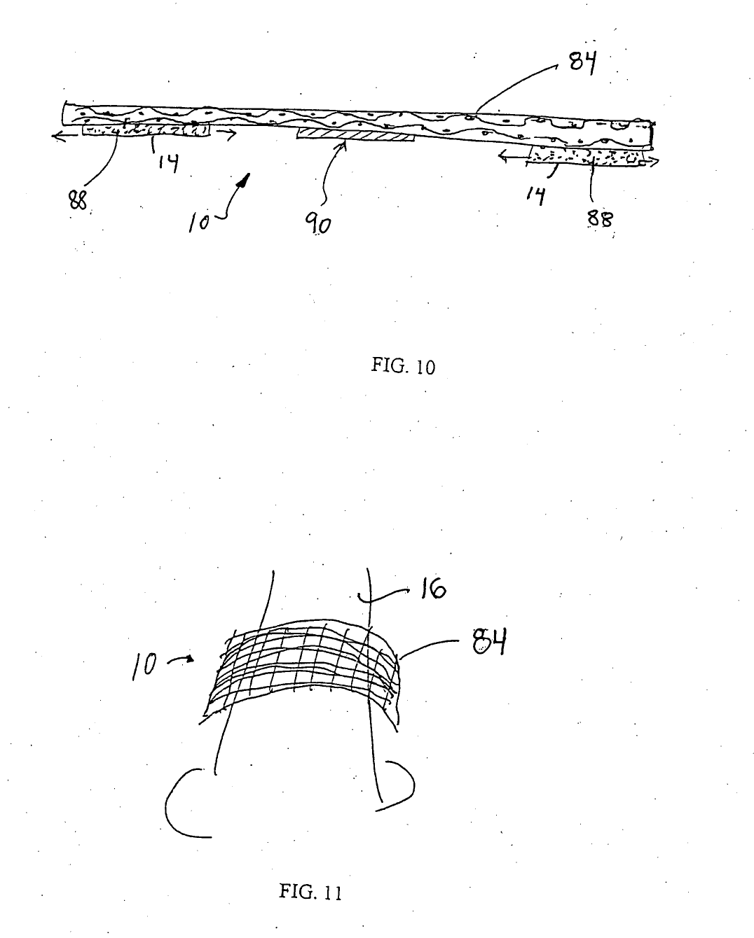

Referring now to FIG. 10 which is an elevational side view of a nasal dilator 10 and wherein the nasal dilator 10 further comprises a pair of adhesive pads 88 which are adhered to user-adjustable, spaced apart locations on one major surface of the nasal dilator 10. In FIG. 10 also depicted is an optional spacer member 90 adhered on one side to the middle region of the nasal dilator 10 to thereby provide an increased dilating lifting force (i.e., increased mechanical advantage) and to provide added comfort to the user by padding the bridge of the nose of the user. The spacer member 90 may be thicker or thinner than the pads 88 as desired and may have adhesive on opposing major surfaces thereof. In FIG. 10, the inherent adjustability of the pads 88 relative to the base 12 is illustrated, but this advantageous adjustability is inherent in several other embodiments of the present invention.

Referring now to FIG. 11 which is an illustration of a nasal dilator 10 which is fabricated in situ on tissue of a user and wherein the nasal dilator 10 preferably comprises a resilient scrim, or mesh, material 84 which is coated with an adhesive material 14. In this embodiment of the present invention, the material 84 is placed adjacent the tissue to be dilated and a preferably fast-drying adhesive solution is applied to the material 84. The adhesive solution may be applied with a brush, dispensed from a tube, an eye-drop type dispenser and/or applied manually. The adhesive solution may contain a scented material and/or menthol, camphor, eucalyptus, or other variety of olfactory or homeopathic materials which may be beneficially inhaled by the subject during application of the adhesive. Such materials tend to be most noticeable while the adhesive solution is drying but additional material may be added to the dilator device 10 to increase the perceptible scent. The scrim material 84 is preferably itself somewhat adhesive, so that it is retained in place during the drying of the adhesive solution. Manual removal of the dilator device 10 may be accomplished with or without use of appropriate solvents, including water, or aided by a thin pliable scraping device (not depicted).

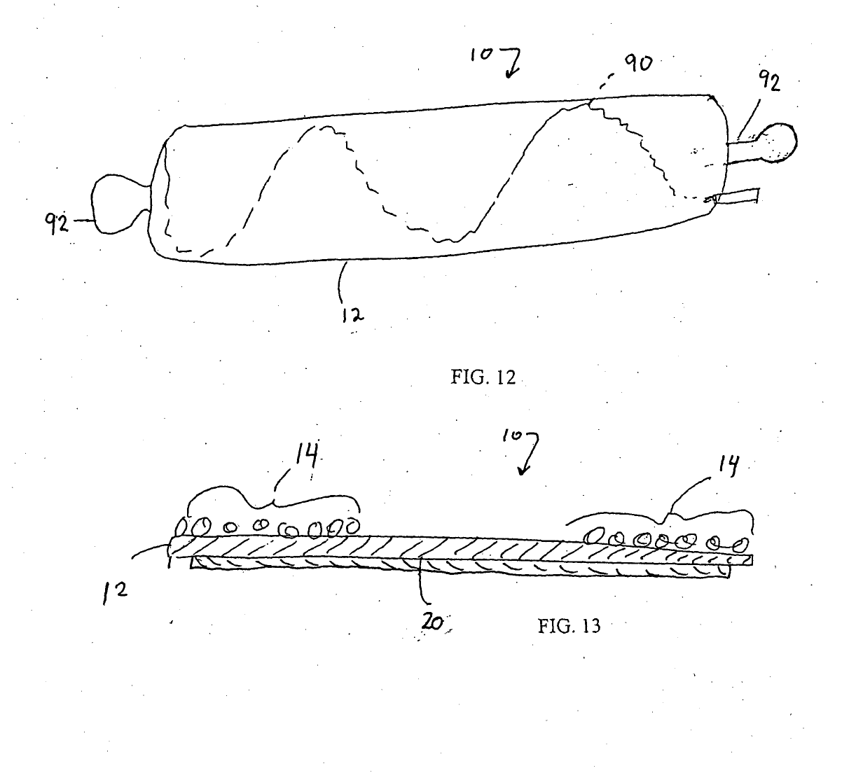

FIG. 12 is a plan view of an elongated nasal dilator displaying several release mechanisms used to promote removal of the nasal dilator from tissue of a user. An elongated nasal dilator apparatus 10 according to this embodiment is first positioned and aligned as desired relative to tissue to be dilated, and then the user releases adhesive material 14 in situ by either abrading a part of the surface of the dilator 10 or removing a serpentine release thread 90 which contacts and breaks open a preferably encapsulated adhesive material 14. Release thread 90 is attached to tab 91 to facilitate thread use. More than one serpentine release thread 90 may be used to vary the amount of adhesive 14 released. A dilator of this embodiment can be reused, That is, if each serpentine release thread 90 contacts a new portion of adhesive which thus provides an increased adhesive bond to the tissue. In lieu of a serpentine release thread 90, a linear perforated portion which corresponds to a thin ribbon, strand, cord, string or filament member and the like may be used. Such a member is used to fully separate the perforated portion and release the adhesive material 14 therefrom when the member breaks a retaining structure, as when the member is pulled across the base 12 of a dilator 10. The adhesive material 14 is preferably micro-encapsulated in individual portions, but may be covered by a layer of film, in sheet form or may be formed in a pattern across a portion of the base 12 of a dilator device 10. The adhesive material 14 may be formed in more than one layer, each layer having at least one release thread 90 (or an equivalent adhesive-releasing mechanism). To reiterate, a basic form of this embodiment includes a dilator device 10 having a thread-like filament 90 disposed therein to promote the rapid release of adhesive material 14 to adhere the dilator to tissue of a user.

Still referring to FIG. 12, another related embodiment of the present invention employs a contrary, yet analogous, use of the release thread 90 just described. That is, use of a thread-like filament 90 (or equivalent) to assist removal of a dilator device 10 from tissue. In this embodiment, a thread, thin ribbon, strand, cord, string or filament and the like 90 is pulled across the plane of a dilator or removed vertically to release a material that inhibits the adhesive material 14 used to adhere the dilator device 10 to tissue of a user. Any material that is compatible with the tissue of the user and that reduces, removes or eliminates the adhesive bond 14 between the dilator device 10 and the tissue may be used. The material is preferably encapsulated or impregnated into the thread 90 (or equivalent) and comes into contact with at least some of the adhesive material 14 of the dilator device 10. Materials such as mineral oil, surfactant, soap, grease, and the like may be used to promote rapid reduction, removal or elimination of the adhesive 14 for the dilator 10.

With continuing reference to FIG. 12, additional release mechanisms which promote separation of the dilator device 10 from the tissue of a user are also depicted. For example an end portion of a resilient member 20 may protrude from one end of an elongated dilator device 10 so that the user many manually grasp the protruding end and lifting or pulling same to remove the dilator device 10 from the tissue. A tab feature 92 may be coupled to an end of the dilator device 10, or may be integrally formed as a part of the base 12 of the dilator device 10. The tab 92 is manually grasped to remove the dilator device 10 from the tissue. Also, the filament 90 may have a tab feature 92 coupled to one or both ends of the filament 90 preferably extending out from the surface of the base member 12 of the dilator device 10 so that the filament structure 90 is more readily accessible to the user. Two such tab features 92 may be affixed to two different filament structures 90 and may be color-coded or provided with other indicia which indicates whether each filament structure 90 provides adhesive or provides adhesive-defeating material when removed from the base member 12 of the dilator device 10.

Referring now to FIG. 13, which is an elevational side view of an embodiment of a nasal dilator 10 according to the present invention wherein a layer of adhesive material 14 is disposed on a side of the nasal dilator 10 and said layer of adhesive material 14 is selectively activated by mechanical action to effectively increase the amount of adhesive available to adhere the nasal dilator 10 to tissue of a user. The adhesive material 14 is preferably a pressure sensitive material that is activated when manually abraded either by an instrument (not depicted) or by direct manual scraping. The adhesive material 14 is analogous to so-called “scratch and sniff” material; that is, material that increasingly is released as the amount of abrasion increases. The material may comprise just adhesive material 14 encapsulated, suspended in a gelatinous material, or contained in or under another material (not shown) that dissolves or is weakened when abraded but may also contain olfactory materials or scents. In this regard, the contents of U.S. Pat. No. 6,248,377 is hereby incorporated in its entirety as if fully set forth herein for its teaching of such scratch and sniff technology. In particular, the scents may comprise odors reminiscent of diverse foods, beverages, flowers, herbs, spices and the like.

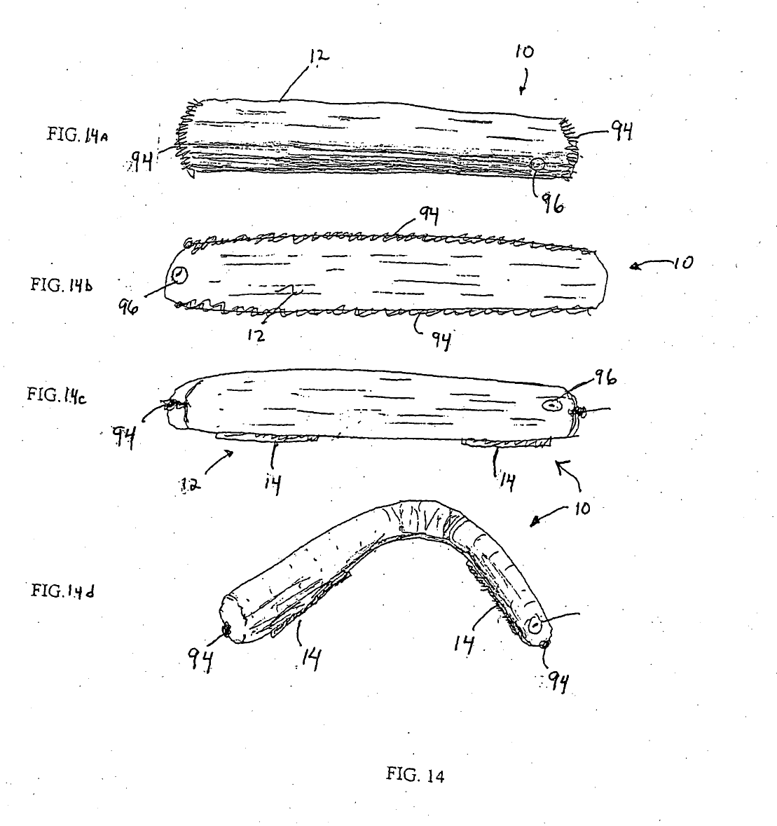

Turning now to FIG. 14a though FIG. 14d, which depict a family of nasal dilators comprised of an elongated unit sealed to ambient conditions and inflated, or filled, with a fluid material—this type of dilator device optionally has a resilient member coupled to an inner and/or an outer surface of the dilator device, a valve for increasing or decreasing the internal fluid pressure and a pair of pads adapted to adhere to a user's nose. In this series of drawings, which depict various views of another embodiment of the present invention, a dilator device 10 which comprises a tube, or balloon-like, shape which is intended to have increased internal pressure relative to ambient pressure and accordingly is sealed to ambient conditions. In FIG. 14a, which is a plan view of a dilator device 10 which has a base member 12 comprising an elongated tube-shaped film or sheet of plastic or suitable polymer material and the like, the opposing ends are sealed using heat and/or pressure to create an air tight seal 94 (said seal depicted with cross-hatching at opposing ends of the elongated dilator). A valve unit 96 may be optionally incorporated into the base member 12 and said valve unit 96 may be manually accessible so that a user may inflate or deflate the interior portion to change the internal pressure of the dilator, and thus, the amount of lifting force imparted by the dilator device 10 to the tissue of the user. In FIG. 14b, which depicts a dilator device 10 which is very similar in construction to the dilator device depicted in FIG. 14a, the opposing ends of base member 12 are already sealed together and a longitudinally oriented seam or seal 94 provides the air tight seal around the periphery of the dilator device 10. Of course, all or a part of the periphery of the dilator 10 may be sealed or only a part of the periphery may be sealed.

FIG. 14c is an elevational side view of the embodiment depicted in FIG. 14a and simply depicts the seam or seal 94, valve 96 and the base member 12 in a fully inflated state. FIG. 14d is nearly identical to the elevational side view of FIG. 14c, but depicts the dilator device 10 as it might appear when affixed to tissue of a user. While not depicted in these drawings, a resilient member 20 may be coupled to or incorporated into the base member 12. Such a resilient member may be coupled to an inner or an outer surface of the dilator 10. As noted with respect to the examples that follow this detailed description, diverse fluids may be used to inflate the base member 12 including viscous and non-viscous fluids, foam or gel may also be used to inflate the base member 12. With respect to the adhesive pads 14 depicted in FIG. 14c and FIG. 14d, such pads may be coupled to a single, common side of the dilator 10 either during initial fabrication or, if desired, later by a user of the dilator 10. The adhesive pads 14 may be coupled to a single side of the dilator 10 or a set of (initially covered) adhesive pads 14 may be coupled at various common radii to accommodate reuse of the dilator 10 by simply uncovering a fresh pair of adhesive pads 14 and applying the dilator 10.

In the embodiments depicted in the FIG. 14 series of drawings, as well as most all other embodiments of the present invention, may be fabricated using luminescent (i.e., glow in the dark) materials. For example the exterior of the dilator 10 may be coated with a suitable luminescent material prior to application, exposed to a light source to activate the material and then applied. In other embodiments, particularly those depicted in FIG. 14 having an interior fluid-filled cavity, such material may be the interior fluid itself or may be individually encapsulated therein. These embodiments, as well as embodiments having retro-reflective material applied or coupled thereto, are intended as primarily ornamental although they may serve a safety purpose for users who are using a dilator 10 at dusk, dawn or during the night. In a similar manner and while not depicted, a dilator 10 may have a radiation absorbing material disposed on the exterior surface. Such radiation absorbing material may help reduce glare for users who are outside in direct sunlight or who are exposed to bright spotlights (e.g., stadium lighting). In addition, for users who are outside in direct sunlight, such radiation absorbing material may help elevate the temperature of the dilator 10 adding comfort to the user (particularly during cold weather). In this embodiment, the dilator device 10 preferably has enlarged opposing ends which couple to the sinus and cheek region of a user. Such enlarged opposing ends do not need to employ a resilient member. A related embodiment is constructed similarly to a face mask used to ward off cold and skin exposure to precipitation and the like.

### EXAMPLES

While several embodiments of the present inventions have been described in detail above with reference to the drawings, the following examples are provided to reinforce the teaching of the present invention without limiting the teaching to any specific illustrated embodiments. However, as will be apparent to the reader most of the examples share analogous structure with the illustrated embodiments. That is, the instant inventions have been described with reference to specific embodiments which are intended as exemplary illustrations and not limiting descriptions of the breadth and scope of the present invention. The following examples as likewise intended to illustrate select several discrete embodiments of the invention to assist comprehension of slightly different embodiments and to promote a fuller understanding of the present inventions.

### Example #1

A elongated nasal dilator apparatus which is first positioned and aligned as desired relative to tissue to be dilated, and then releasing adhesive material in situ by either abrading a part of the dilator or removing a serpentine release thread which contacts and breaks encapsulated adhesive material. More than one serpentine release thread may be used to vary the amount of adhesive released. If desired this embodiment allows reuse of a dilator device if each serpentine release thread contacts a new portion of adhesive which provides an effective adhesive bond to the tissue. In lieu of a serpentine release thread, a linear perforated feature may comprise a thin ribbon, strand, cord, string or filament and the like and same may be pulled across the plane of a dilator or may be removed vertically. The adhesive material may be encapsulated in individual portions, may be covered in a layer of film or in sheet form or may be formed in a pattern across a portion of a dilator. The adhesive material may be formed in more than one layer, each layer having at least one release thread (or an equivalent adhesive-releasing mechanism). To reiterate, a basic form of this embodiment includes a dilator device having a thread disposed therein to promote the rapid release of adhesive material used to adhere the dilator to a user.

### Example #2

This embodiment is related to Example #1 inasmuch as it relates to use of a directly contrary, yet analogous, use of the release thread of Example #1; that is, use of a thread (or equivalent) to assist removal of a dilator from tissue. In this embodiment, a thread, thin ribbon, strand, cord, string or filament and the like is pulled across the plane of a dilator or removed vertically to release a material that inhibits the adhesive material used to adhere the dilator to a user. Any material that is compatible with the tissue of the user and that reduces, removes or eliminates the adhesive bond between the dilator and the tissue may be used. The material is preferably encapsulated or impregnated into the thread (or equivalent) and comes into contact with at least some of the adhesive material of the dilator. Materials such as bubbles of oil, surfactant, soap, grease, and the like may be used to promote rapid reduction, removal or elimination of the adhesive for the dilator.

### Example #3

A continuous substantially planar dilator segment or strip of dilator material comprised of many single extruded dilator devices that may be applied by adhering a first side, then twisting the dilator device one or more full turns around a twist section and then adhering the second side. The twist section of the dilator may be covered or uncovered. If covered, the covering is preferably a material that encircles the twist section of the dilator and provides padding to relieve stress to the tissue that may occur if the twist section directly rests on the tissue. The twist section may be fabricated to promote an preselected topography to the twist section. That is, the twist section may be formed of a material having different resiliency than other parts of the dilator and/or may be perforated, folded or provided with creases so that after the dilator is twisted by a user the twist section assumes a relatively smooth cross section. A dilator constructed and used according to this example may have indicia on surfaces thereof, so that when twisted, numerals or other indicia that were initially visible are covered by dilator material in the twist section of the dilator. For example, the numerals, “1, 2, 3” may be initially visible, but after a first turn the “1” is covered or folded, after a second turn the “2” is covered or folded, and after a third turn the “3” is covered or folded over. Preferably, the twist section is fabricated with a resilient member embedded therein so that regardless of the number of turns, or twists, of the dilator, the dilator still provides a restoring force from its normal planar (or linear) condition. Most preferably, the resilient member comprises a resilient scrim, mesh or net composed of many individual resilient filaments or threads and the like. In this embodiment, the length of the dilator device is typically reduced by each turn of the dilator.

In a further related embodiment, a single resilient member is disposed in a pocket or sleeve or the dilator and the resilient member essentially floats in said pocket or sleeve when the body of the dilator is twisted and thus the resilient member does not twist. Preferably, such a single resilient member is elongated and has a cross section that is round and the resilient member may comprise a hollow tube. Of course the resilient member may have diverse symmetric and asymmetric cross sectional shapes, including having two or more major planar surfaces.

### Example #4

In this example, a dual-use dilator is fabricated with adhesive that is selected by the user or comprises a skin-type specific adhesive formulation which is particularly useful for adhering the dilator to tissue that is extraordinarily wet, greasy or dirty. In one form of this embodiment, a dilator device has two release liners, one for each side of the dilator and each side of the dilator has a different strength adhesive disposed thereon. Of course, the same adhesive formulation may be disposed on both side of the dilator device as well. The exterior packaging of such a dual-use dilator preferably indicates on the release liner the type of adhesive, or the relative strength of the adhesive formulation, present under the release liner. This dual-use dilator may be simply reused (once) by the user or discarded after the initial use. Of course, this form of the present invention may be used merely to give the user a second try at correctly applying the dilator device to tissue of the user, if the first attempt is not satisfactory.

A related embodiment relates to stacking such dilator devices to create greater lifting force to the tissue and thus promote ease of respiration of the user. For example, if an initial dual-use dilator is applied to dilate tissue, a second (“standard” one-sided) or additional dual-use dilators may be stacked to increase the lifting force, or to distribute lifting force over a greater area of tissue if adhered slightly offset from a prior dilator device.

### Example #5

A family of dilator devices fabricated of a relatively fast-drying gel-, liquid- or fluid-based material which is preferably applied directly to tissue to be dilated. The material is coated, layers, deposited, sprayed or brushed onto the surface of each side of the nose of a user. The dilator devices of this embodiment preferably includes an adhesive bridge structure which is placed over the bridge of the user's nose prior to adding the fast-drying materials. Thus, the desired tissue lifting force is generated and sustained as the material dries (i.e., contracts and tightens as it dries). The bridge structure may be devoid of adhesive or have a modicum of adhesive to keep the bridge structure in place prior to adding the materials on each side of the bridge structure. While a dilator device formed as described will provide some lifting force, the bridge structure amplifies and directs the lifting force imparted to the tissue. A wide variety of bridge structures may be used to create the desired dilation of tissue, but a relatively low profile bridge structure is preferred that largely conforms to the bridge of the user's nose and which compresses slightly as the material dries. The lateral ends of the bridge structure preferably gradually slope continuously to a thin lateral periphery portion of the bridge structure. In these embodiments, a plurality of oriented temperature-sensitive fibrils may be incorporated into the material directly or may be provided on the exposed surface of the bridge structure. The bridge structure may be formed entirely of a resilient scrim, mesh or net-type web of individual resilient members. These oriented temperature-sensitive fibrils are intended to add strength to the material when dried and to promote tension-bearing performance in said dried material. The material itself should be non-toxic to the user and should contract significantly from the liquid (or gel) state to the solid state of the material. The dilator devices of this embodiment may be tinted to match the skin color of the user or otherwise colored for effect (e.g., matching school or team color scheme) with non-toxic coloring agents, colorants, pigments, dyes and the like.

### Example #6

Another embodiment relates to a family of dilator devices having at least one biosensor embedded or incorporated into the dilator device to detect a physical parameter of the user of the dilator. In this embodiment, a dilator device adapted to sense the blood pressure, temperature, rate of respiration, heart rate of the user or to sense ambient “air quality” (e.g., presence of pollen) or the user's exposure to airborne chemical, radioactive, biological, acidic or basic (i.e., high pH) materials. For example, a material that changes color or conducts electricity in the presence of such materials may be disposed on or integrated into a dilator device and such color change or electrical stimulation provides a cue so the subject may act accordingly.

### Example #7

Another embodiment of dilator devices constructed according to the present invention includes a family of elongated dilators that are easy to apply, use and remove that are formed entirely of a non-irritating, resilient adhesive material. These dilator devices are preferably extruded as a unitary structure formed of homogenous, resilient adhesive material. In this embodiment, the entire dilator device acts a resilient member that provides a restoring force that biases the dilator device to return to its original substantially planar configuration. The material is preferably homogenous having adhesive properties that provide adequate adherence between the dilator and the tissue while at the same time possessing resiliency (i.e., a restoring force biasing the dilator back to a substantially flat configuration. As a result, dilator devices fabricated according to this embodiment may be reused by simply removing a layer of material from the surface to expose new material which may be adhered. This type of dilator device may be packaged in individual segments for use but is preferably dispensed as a continuous roll of resilient dilator material having a single separation, or release, liner between successive windings, or loops, of material. The user simply removes a segment of material by cutting, tearing or severing the segment from the roll of material, removing the separation liner and affixing a first end of a first side to tissue to be dilated, slightly stretching the dilator segment (e.g., over the nose), and affixing the second end of the first side to the tissue. Alternatively, the separation liner may be perforated at a short interval so that the user may remove the separation liner from only the first end and the second end thereby simultaneously slightly increasing comfort to the user and the effective lifting force of each end of the dilator segment. In addition, the dilator segment itself may be periodically perforated so that the user may dispense and use a variety of length dilator segments. Of course, the periodic perforations may be surface cuts, slices or holes formed in the dilator material so long as same do not compromise the structural integrity of the resulting dilator device. A related form of this embodiment is provided wherein the entire length of the “raw” dilator is perforated, preferably after being extruded, with a variety of apertures to decrease the weight of each resulting dilator segment and to increase ventilation to the underlying tissue when in use. The perforations may be formed mechanically, using tooling or fluid to punch or cut linear apertures through the dilator material or may be formed prior to extruding by injecting ambient air, or other relatively inert gaseous material, into the material prior to extruding same. As with other embodiments of the present invention, these embodiments may be colored, tinted, or shaded as desired prior to extrusion for a particular application. These types of elongated dilator devices may be extruded in either the longitudinal axis or the lateral axis to form the elongated dilator devices of the present invention. In the event that a dilator segment separated from a roll of dilator material is curled because it was formed and/or stored in a roll, the user may simply apply the convex side of the curled dilator segment to the tissue (and thus provide a slightly increased restoring force). Alternatively, the user may remove such curled dilator segments and place them on a flat surface (with corresponding separation liner material) until the curl is reduced. Some selected materials may be heated briefly to more rapidly reduce or eliminate the curl of a dilator segment. Instead of essentially homogenous adhesive and resilient material used in the extrusion process a monolithic resilient member, scrim, mesh, fabric or netting may be co-extruded with an adhesive material with the proviso that any later apertures, cuts, tears or slices should be accomplished with an eye toward retaining the resiliency of the dilator while also allowing the user to decide what length of dilator segment to use.

### Example #8

Another embodiment of dilator devices according to the present invention include a family of “pacing” dilator devices. That is, dilator devices that function somewhat similarly to a metronome providing biofeedback to the user related to the rhythm and rate of respiration. This family of dilator devices has many forms, but one preferred form involves use of a substantially flat elongated resilient member that remains substantially flat after being applied to dilate tissue on opposing sides the nose of a user. In this form of the present invention, the dilator device is constructed to emit a vibration in response to breathing conditions of the user. Thus, in the one preferred embodiment, the vibration is emitted by the resilient member when the user inhales or exhales, thus slightly distorting the resilient member from its substantially flat configuration. The noise may be audible or may be passively transmitted through the bone structure of the user and sensed as a slight vibration originating near the nose of the user. An alternate form of this invention implements a hinged resilient member—having a small range of motion for said hinge and preferably including a detent and a corresponding boss member—so that when the hinge is activated, the boss enters the detent (and “clicks”) and then exits the hinge (providing another “click”). Another form of this invention uses an elongated resilient member having opposing major planar surfaces that is bowed due to compression forces acting on the ends of the resilient member. The effect is similar to a batten constrained in a pocket of a sail on a sailboat. That is, each time a force impinges upon the convex side of the resilient member of a first state, the resilient member “snaps” to the opposite configuration of a second state (i.e., convex side becomes concave side and vice versa). When balanced with a relatively weak compressive force, the resilient member reciprocates between the first state and second state. The preferred form of this embodiment of the invention thus provides biofeedback to the user as the resilient member transitions from the first state to the second state. The resilient member may be adhered to the bridge of a user's nose or simply affixed to the user's nose thus forming a pivot location at the bridge of the nose and wherein each end of the resilient member is coupled to the tissue via a length of generally non-elastic material having an attachment coupling at the end. The attachment coupling may comprise a suction cup, a patch of adhesive or adhesive disposed upon the interior cavity of said suction cup and the like. Of course, the use of a resilient member that is maintained in a state of compression may be used without practicing the “pacing” function described above, as such a resilient member will still provide the required lifting forces to the tissue regardless whether or not the resilient member transitions between two energy states.

### Example #9

A further embodiment of the present invention comprises an elongated fluid-filled vessel having adhesive or suction-type material on a portion of the side thereof. The fluid is preferably ambient air, manually injected at an elevated pressure relative to ambient air pressure and the vessel is preferably a resin-based or plastic tube. The tube may be filled with fluid during initial fabrication and sealed, or may be filled with fluid by the user just prior to use of the dilator device in which case a manually operable valve is preferably fitted to one side thereof. Said valve may extend from, or be insertable into, the body of the vessel. The dilator devices constructed according to this form of the invention may also include an elongated resilient member that continually provides a restoring force when bent from an original configuration. However, provided that adequate fluid pressure is contained inside the vessel, relative to ambient pressure, no such resilient member is required in order for a dilator device so constructed to provide an adequate lifting force to dilate nasal tissue.

One advantage of this embodiment, is that the dilator device may be shipped in quantity in a very compact container to distributors or end user consumers. Another advantage of this embodiment relates to the fact that such a dilator device inherently increases its interior fluid pressure when subjected to increased heat. Thus, when a user exercises using this form of the invention and is need of additional respiration volume, the dilator device automatically responds with increased lifting force and therefore additional respiration volume.

A variety of ways of introducing increased fluid pressure to the vessel may be used; such as via use of: a manual pump, a source of compressed fluid, a bellows, a source of heat, and the like. A manual pump adapted for use in conjunction with this embodiment may be incorporated into the dilator device or may be a remote device capable of delivering pressurized fluid, and is preferably incorporated into packaging for the dilator device. If the pump is incorporated into the device, then a single fluid chamber coupled to a valve is preferably provided sized to be actuated by a finger of the user.

The shape of the fluid-filled elongated vessel according to this embodiment of the present invention may take many forms. In cross section the vessel may be a round, a geometric or a polygon shape.

A reusable form of this embodiment has a series of individual adhesive portions that are disposed longitudinally (or in the form of discrete adhesive patches) and thus may be individually revealed for a single use of the dilator. In this form of the invention, a series of adjacent elongated patterns of adhesive are each covered with a release liner. Each release liner may be individually removed so that the dilator may be used once for each release liner removed.

The fluid may comprise a compound gaseous fluid such as air or a single gas or combination thereof. The fluid may comprise liquid such as water or aqueous-based formulations or non-aqueous-based formulations selected because of their ability to expand when subjected to heat or to absorb heat without expanding. The dilator vessel may comprise a combination of gaseous and liquid fluids contained in separate or common sealed compartments forming a part of the dilator vessel.

If one or more elongated resilient members are included in this embodiment of the invention, they may be coupled to the interior or exterior of the dilator vessel. In addition to or in lieu of such a resilient member, the vessel itself may be comprised of materials having different characteristics, such as a low modulus of elasticity for a portion disposed adjacent to the tissue to be dilated and having a larger modulus of elasticity for other portions of said vessel.

Such differing characteristics may be used to indicate an overpressure condition or to indicate that a maximum usable fluid pressure have been reached for a given dilator device. A valve may be a specially adapted valve requiring a complementary inflation stem or may comprise a flap of flaps of material which are adhered in an airtight seal.

This embodiment may be cast in a form useful in treating trauma or pain in the tissue to be dilated by using a cold or heat-absorbing fluid to fill the dilator device. By simultaneously applying what essentially amounts to an ice pack over the nose of a user and increasing respiration volume the user may find more rapid relief from the trauma or pain in the tissue.

To reduce the lifting force or prepare the dilator device for removal, a release valve may be opened or the fluid seal of the dilator device may be broken thereby equalizing the fluid pressure to ambient conditions and reducing the lifting force provided by the pressurized fluid.

If the fluid contains one or more agents or ingredients that reduces or dissolves any adhesive used to adhere the dilator to the tissue, then simply piercing the vessel and releasing some of the fluid will cause the dilator device to stop adhering to the tissue and eases removal from the tissue.

A related form of this embodiment involves first filling the fluid vessel with a material that may be readily dispensed therefrom, having the user empty the material, inflating the vessel and applying the dilator to tissue. The material may comprise a lozenge, gum, tablets, pills, powder, leaves, and liquid forms of same and the like. Liquid may include dissolved medication such as aspirin, ibuprofen and the like or may include protein-fortified formulations and the like intended to increase endurance and stamina in the user. The vessel may include sun blocking agents or so-called sun screen in a primary or secondary compartment of said vessel.

The material used to construct the vessel may be sufficiently elastic to permit one portion to be adhered to tissue while an other portion is provided with tension adequate to elongated the vessel prior to adhering the other portion to tissue. Thus, an additional amount of lifting force may be applied to the tissue. Adhesive material may be disposed circumferentially around the base member of the dilator and a plurality of release liners may cover different adhesive portions so that the dilator device may be reused by simply revealing additional adhesive and applying the dilator to the tissue to be dilated.

### Example #10

A dilator formed of perforated or porous materials for single use and multiple use (pockets in materials to receive resilient members) and/or non-adhesive dilators with suction cups (non-adhesive) or with a series of suction cups and a small amount of adhesive applied each time the dilator is used. In this embodiment of the present invention, the base member is preferably washable and may be reused with a variety of different resilient members each providing a different magnitude lifting force. To simplify removal of such suction cups, manually accessible tabs may be coupled to an edge of one or more suction cups as is known and used in the art.

The foregoing descriptions and illustrations are intended to reveal the true scope and spirit of the present inventions and should not be interpreted as limiting, but rather as illustrative of the inventive concepts and techniques thereof. The claims, when properly interpreted provide the true and complete metes and bounds of the present invention and they alone should be used to gauge the breadth and scope of the teaching hereof. Of course, those of skill in the art to which the present inventions are directed will appreciate that insubstantial changes, modifications and alterations of the present disclosure may be made and each such insubstantial change, modification and alteration are intended to be fully covered hereby.

1. A nasal device for communicating physiologically-related information to a user, said device comprising:
a flexible strip of material having a first end region and a second end region and an intermediate region, said flexible strip adapted to be adhesively secured to nasal surfaces of a user, and
a signal device which senses physiological information of the user while disposed upon the nasal surfaces and which communicates information to the user related to sensed physiological information via one or more of: acoustic vibrations, tactile contacts and light emissions.

2. The nasal device of claim 1 wherein the signal device includes a plurality of sensors and a plurality of communication means.

3. The nasal device of claim 1 wherein the signal device is disposed at the first end region, the second end region, or the intermediate region.

4. The nasal device of claim 1 wherein the signal device receives information from an external source and utilizes this information to alter an output process.

5. The nasal device of claim 1 wherein the signal device transmits information to a remote transceiver for subsequent signal processing.

6. The nasal device of claim 5 wherein the signal device transmits via radio-frequency or infra-red communication.

7. The nasal device of claim 1 wherein the signal device includes memory means for storing information.

8. The nasal device of claim 1 wherein the signal device includes a timing means for controlling application of a tactile contact.

9. The nasal device of claim 8 wherein the tactile contact is a periodic vibration.

10. The nasal device of claim 1 wherein the signal device is integrated within flexible strips of material.

11. A nasal device of claim 1 further comprising:
a resilient member coupled to the flexible member which provides a lifting force to portions of the nasal surfaces of the user.

12. A nasal device comprising:
a flexible strip of material adapted to be placed on nasal surfaces of a user;
an adhesive layer coupled to the flexible strip of material for securing the flexible strip to the nasal surfaces;
a sensor means for sensing physiological information of the user at the nasal surfaces; and
a tactile vibration means for communicating tactile information to a user at the nasal surfaces, said tactile information being related to the physiological information.

13. The nasal device of claim 12 wherein the sensor means includes an acoustic sensor for sensing vibrations of the nasal surfaces related to the user's breathing pattern.

14. The nasal device of claim 12 further comprising:
a resilient member coupled to the flexible strip of material which provides a lifting force to the nasal surfaces of the user.

15. A nasal device comprising:
a flexible strip of material adapted to be placed on nasal surfaces of a user;
an adhesive layer coupled to the flexible strip of material for securing the flexible strip to the nasal surfaces; and
a tactile vibration means for communicating tactile information to a user at the nasal surfaces.

16. The nasal device of claim 15 further comprising:
a sensing means for sensing physiological information of the user.

17. The nasal device of claim 16 further comprising:
a microprocessor for receiving a signal from the sensing means and for controlling a function of the tactile vibration means.

18. The nasal device of claim 17 further comprising a communications unit for communicating information to and from the nasal surfaces of the user.

19. The nasal device of claim 15 further comprising:
a resilient member coupled to the flexible strip of material which provides a lifting force to the nasal surfaces of the user.

20. The nasal device of claim 16 wherein the tactile vibration means provides a periodic tactile transmission to the nasal surfaces of the user.

21. A method of receiving information related to a user's physiological condition, said method comprising the steps of:
adhering a flexible strip of material at nasal surfaces of a user, said flexible strip of material being coupled to a sensor and a controllable tactile vibrator;
sensing a physiological parameter of the user at the nasal surface of the user; and
communicating tactile information to a user at the nasal surfaces via the tactile vibrator.

22. The method of claim 21 further comprising the steps of:
performing a data computation on information received from the step of sensing.

23. The method of claim 22 further comprising the steps of:
receiving commands from an external source, and adjusting a tactile vibrator operation in response to the received commands.

24. The method of claim 22 further comprising the steps of:
transmitting information relating to information received from the step of sensing.

25. The method of claim 21 wherein the physiological parameter includes one or more of: a heart rate parameter, a blood pressure parameter, or a temperature parameter.

26. A nasal dilator device, comprising:
an elongated body portion of a flexible material;
a layer of adhesive material disposed on a side of the body portion; and
means for producing a vibration which when coupled to a portion of tissue of a user is sensed by the user of the dilator device.

27. A nasal dilator device, comprising:
an elongated body portion of a flexible material;
a layer of adhesive material disposed on a side of the body portion; and
a vibrator coupled with the body portion which transmits a user-discernible force to a portion of tissue of a user.

28. A dilator device having a reusable resilient member, comprising;
an elongated member having a pair of pockets disposed at each end of the elongated member wherein each of said pair of pockets has an opening and each opening generally faces the other opening;
at least two areas of adhesive material disposed on a portion of each end of the elongated member; and
an elongated resilient element coupled to the elongated member and having a first end of the elongated resilient element disposed in a first of said pair of pockets and a second end of the elongated resilient element disposed in a second of said pair of pockets.

29. A dilator device according to claim 28, wherein the elongated member is formed of a perforated or porous material.

30. A dilator device according to claim 28, further comprising at least a pair of structures adapted to retain the elongated resilient element.

31. An adjustable force dilator device, comprising:
an elongated strip of elastic material having an adhesive material disposed on at least one major surface of the elongated strip and wherein said elongated elastic strip has an intermediate portion adapted to be twisted about an longitudinal axis of the elongated strip so that the at least one major surface may be adhered to a first portion of tissue and manually twisted an integer number of full turns about the longitudinal axis and then the at least one major surface may be adhered to a second portion of tissue.

32. An adjustable force dilator device according to claim 31 wherein the adhesive material is disposed on a first major surface and an opposing second major surface of the elongated strip and the elongated strip is twisted an integer number of half turns about the longitudinal axis before being adhered to the first portion of tissue and the second portion of tissue.

33. An adjustable force dilator device according to claim 31 further comprising:
an elongated resilient member disposed within the elongated dilator device.

34. A nasal dilator device comprising:
an elongated flexible body including a sealed chamber containing a fluid; and
an adhesive material disposed on the body for securing the body onto nasal surfaces of a user.

35. The nasal dilator device of claim 34 wherein the fluid is air.

36. A nasal dilator device comprising a fluid-filled elongated vessel adapted to conform to nasal surfaces of the user and to be secured to the nasal surfaces via an adhesive element.

37. An apparatus comprising:
a nasal dilator having resilient properties and adapted to be adhesively bonded to nasal surfaces of a user; and
thread means integrated into the nasal dilator for selectively decreasing the adhesive bond between the nasal dilator and the nasal surfaces of the user and facilitating removal of the nasal dilator by the user.
